(19) United States
(12) Patent Application Publication
(10) Pub. No.: US 2004/0132645 A1
(43) Pub. Date: Jul. 8, 2004

(54) Methods for modulating effects of radiation in a subject

(76) Inventors: Susan J. Knox, Stanford, CA (US); Shoucheng Ning, Stanford, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/336,238

(22) Filed: Jan. 3, 2003

(51) Int. Cl. A61K 38/22

(52) U.S. Cl. 514/12

(57) ABSTRACT

The invention features methods and compositions for modulating the effects of radiation in a subject. In one embodiment, the invention features methods for enhancing radiosensitivity of a tumor in an anemic subject by administration of erythropoietin (EPO), particularly a hyperglycosylated EPO isoform such as ARANESP™. In general, the invention provides methods for potentiating the effects of radiation therapy in a subject without the need for correction or complete correction of anemia in the subject prior to initiation of radiation therapy. The invention further provides for improved recovery from bone marrow suppression (including non erythroid cell types) and improved overall health of the subject. In another embodiment the invention features methods for treatment of an irradiated subject by accelerating recovery of bone marrow cells (including non-erythroid cell types) in an irradiated subject or mitigating the effects of radiation upon such cells.

### FIELD OF THE INVENTION

The present invention is in the field of cancer therapy, particularly radiotherapy, and therapy of illness associated with whole body irradiation.

### BACKGROUND OF THE INVENTION

Erythropoietin (EPO) is a glycoprotein hormone involved in the maturation of erythroid progenitor cells into erythrocytes. It is essential in regulating levels of red blood cells in circulation. Naturally occurring erythropoietin is produced by the liver during fetal life and by the kidney of adults and circulates in the blood and stimulates the production of red blood cells in bone marrow. Anemia can be a consequence of renal failure due to decreased production of erythropoietin from the kidney, and can also result from intentional or accidental exposure to radiation. In the latter case, anemia can be accompanied by suppression of bone marrow-derived cells, with reduction in white blood cells, including neutrophils. Recombinant EPO is effective when used in the treatment of anemia resulting from chronic renal failure.

Anemia is also common in clinical oncology. It can occur secondary to the cancer itself and is also a common complication in patients undergoing cancer therapy, including chemotherapy and radiation therapy. The use of EPO, as well as the hyperglycosylated EPO isoform ARANESP™, in treatment of anemia in cancer patients has been described (see, e.g., U.S. Pat. No. 4,745,099; for reviews see, e.g., Jung et al. 2002 Cancer Pract 10(6):327-30; and Crawford 2002 Oncology (Huntingt) 16(9 Suppl 10):41-53; Clark et al. 2002 BMC Cancer 2(1):23′ Itri, Semin Oncol. 2002 June;29(3 Suppl 8):81-7; Smith 2002 Curr Opin Hematol 9(3):228-33; and Demetri 2001 Br J Cancer. 84 Suppl 1:31-7). Administration of EPO has also been suggested to improve the quality of life of cancer patients (see, e.g., U.S. published application no. 20020169129; Del Mastro et al. 1998 The Oncologist 3:314-318; Taylor 2003 Med Hypotheses 60(1):89-93; Littlewood et al. 2002 Lancet Oncol. 3(8):459-60; Littlewood et al. 2002 Semin Oncol 29(3 Suppl 8):40-4).

Anemia has been implicated as an indicator of poor therapeutic outcome for cancer patients undergoing radiation therapy. Many researchers have hypothesized that tumor hypoxia contributes to radioresistance of tumors (see, e.g., Moulder et al. 1984 Int J Radiat Oncol Biol Phys 10:695-712; Rockwell et al. 1990 Int J Radiat Oncol Biol Phys 19:197-202; Rizzo 2001 Hematology 2001:10-130). Further, several studies have implied that hypoxia is relevant in the clinical situation (Oergaard 1989 Int J Radiat Biol 56:810-811; Nordsmark et al. 1996 Radiother Oncol 41:3139; Hockel et al. 1993 Radiother Oncol 26:45-50; Hockel et al. 1996 Cancer Res 56:4509-4515; and Kaanders et al. 1998 Radiother Oncol 48:115-122). In addition, EPO receptor expression is altered in several cancer cell types (see, e.g., Leyland-Jones et al. 2002 Semin Oncol 29(3 Suppl 11):145-54).

In order to improve the response of tumors to radiation therapy, several studies have implied or suggested that correction of anemia in patients prior to initiating radiation therapy or chemotherapy is warranted. See, e.g., Stuben et al. 2001 J Cancer Res Clin Oncol 127:346-350; Henke et al. 1999 Radiother Oncol 50:185-190; Antonadou et al. 1998 Radither Oncol 48(suppl):483; Thews et al. 2001 Cancer Res 61:1358-1361; Golab et al. 2002 Clin Cancer Res 8:1265-1270; Henke 2001 Onkologie 24:450-454; Henke et al. 2000 Int J Radiat Oncol Biol Phys 48:339-345; Glaser et al. 2001 Int J Radiat Oncol Biol Phys 50:705-715; Kelleher et al. 1996 Cancer Res 56:4728-4734; and Kelleher et al. 1998 Strahlenther Onkol 174 Suppl 4:20-3 (the latter noting that levels of radiosensitivity found in non-anemic animals was not achieved).

Under these suggested therapies, initiation of radiation therapy must be delayed in order to allow for recovery of the patient from anemia. Such delay has certain disadvantages, including increased visits to the clinic, and increased costs. Furthermore, delay in initiating therapy can be, in certain settings, critical to outcome (e.g. the tumor can grow during the time it takes to correct the anemia). Thus there remains a need in the field for methods of treating tumors in anemic subjects, and improving the sensitivity of tumors in anemic subjects to radiation. The present invention addresses these needs.

### SUMMARY OF THE INVENTION

The invention features methods and compositions for modulating the effects of radiation in a subject. In one embodiment, the invention features methods for enhancing radiosensitivity of a tumor in an anemic subject by administration of erythropoietin (EPO), particularly a hyperglycosylated EPO isoform such as ARANESP™. In general, the invention provides methods for potentiating the effects of radiation therapy in a subject without the need for correction or complete correction of anemia in the subject prior to initiation of radiation therapy. The invention further provides for improved recovery from bone marrow suppression (including non erythroid cell types) and improved overall health of the subject. In another embodiment, the invention features methods for treatment of an irradiated subject by accelerating recovery of bone marrow cells (including non-erythroid cell types) in the irradiated subject or mitigating the effects of radiation upon such cells.

Thus, in one aspect the invention features methods for enhancing radiation sensitivity of a tumor in an anemic subject, by administering to the anemic subject an amount of erythropoietin (EPO) effective to enhance radiosensitivity of the tumor relative to the absence of said administering; wherein radiation sensitivity of the tumor is enhanced prior to or without correction of anemia in the subject. In a related aspect, the invention features methods for treating a tumor in an anemic subject, the method comprising administering to an anemic subject having a tumor an amount of erythropoietin (EPO) effective to enhance sensitivity of the tumor to radiation relative to the absence of said administering; and irradiating the tumor of the anemic subject, wherein the tumor in the subject is treated.

In related embodiments, the EPO is a recombinant EPO having an increased number of sialic residues relative to naturally-occurring EPO. In one embodiment, the EPO is darbepoetin alfa. In further related embodiments, the EPO is administered weekly, and in still further related embodiments, the EPO is administered in a dose of from about 0.5 mcg/kg body weight to 4.5 mcg/kg body weight. In further related embodiments, the EPO is administered once every two weeks, and in still more related embodiments, the EPO is administered in a dose of from about 1.0 mcg/kg body weight to 10 mcg/kg body weight.

In other embodiments, EPO is administered prior to irradiating the tumor. In other embodiments, the tumor is irradiated prior to or at the time of EPO administration. In an embodiment of specific interest, the tumor is a solid tumor or a lymphohematopoietic tumor.

In another aspect, the invention features methods for treating a subject suffering from or at risk of bone marrow suppression associated with radiation exposure, the method comprising administering to the subject an amount of erythropoietin (EPO) effective to enhance recovery of red and white blood cell levels in the subject, wherein administering is effective to mitigate the effects of radiation upon bone marrow suppression in the subject. In related embodiments, the radiation exposure is whole body irradiation. In further related embodiments, the EPO is a recombinant EPO having an increased number of sialic residues relative to naturally-occurring EPO, such as darpoictin alfa.

In further embodiments, the subject has been exposed to radiation, and EPO is administered within about 7 days after radiation exposure. In related embodiments, EPO administration is effective to increase white blood cells levels within about 80% of normal levels for the subject, and/or effective to increase neutrophil levels within about 80% of normal level for the subject.

In another related embodiment, the subject is at risk of radiation exposure, and EPO is administered within about 1 day prior to radiation exposure. In further related embodiments, EPO administration is effective to mitigate the effects of radiation exposure such that white blood cell levels in the subject are maintained at least about 50% of normal white blood cell levels and/or mitigate the effects of radiation exposure such that neutrophils levels in the subject are maintained at least about 50% of normal neutrophils levels.

A distinct advantage of the methods of the invention is that radiation therapy need not be delayed in order to allow for time for correction of anemia in the subject.

Another advantage is that the methods of the invention provide for improved overall health of the subject, e.g., correction of and/or delay in whole body irradiation effects; facilitating recovery of bone marrow suppression that results from radiation therapy (e.g., enhanced white blood cell recovery, weight gain, general well-being, etc.).

Still another advantage is that EPO can be administered just prior to, at the time of, or at a time after intentional or accidental exposure of a subject to radiation, particularly whole body radiation, to facilitate recovery of bone marrow-derived cells (including white blood cells such as neutrophils) after exposure and/or to mitigate the effects of such exposure upon such cells.

### BRIEF DESCRIPTION OF THE DRAWINGS

FIG. 1 is a graph showing the effect of total body irradiation (TBI) upon hemoglobin levels in C3H mice.

FIG. 2 is a graph showing the effect of TBI upon hematocrit levels in C3H mice.

FIG. 3 is a graph showing the effect of TBI upon red blood cell (RBC) levels in C3H mice.

FIG. 4 is a graph showing the effect of TBI upon white blood cell (WBC) levels in C3H mice.

FIG. 5 is a graph showing the effects of ARANESP™ on hemoglobin levels of C3H mice having TBI-induced anemia.

FIG. 6 is a graph showing the effects of ARANESP™ on hematocrit levels of C3H mice having TBI-induced anemia.

FIG. 7 is a graph showing the effects of ARANESP™ on red blood cell levels of C3H mice having TBI-induced anemia.

FIG. 8 is a graph showing the effects of ARANESP™ on white blood cell levels of C3H mice having TBI-induced anemia.

FIG. 9 is a graph showing the effects of ARANESP™ on neutrophil levels of C3H mice having TBI-induced anemia.

FIG. 10 is a graph showing the effects of ARANESP™ on lymphocyte levels of C3H mice having TBI-induced anemia.

FIG. 11 is a graph comparing tumor growth in TBI-induced anemic mice either with or without ARANESP™ treatment.

FIG. 12 is a graph showing the effect of ARANESP™ on radiation response of tumors in TBI-induced anemic mice.

FIG. 13 is a graph showing the effect of ARANESP™ on radiation response of tumors in TBI-induced anemic mice.

FIG. 14, is a graph showing the body weight of TBI-induced anemic mice having tumors and treated with fractionated radiation therapy, with or without ARANESP™.

### DEFINITIONS

“Erythropoietin” or “EPO” as used herein includes naturally occurring perythropoietin, urinary derived human erythropoietin, erythropoietin isoforms, as well as non-naturally occurring polypeptides (e.g., recombinant or synthetic) having an amino acid sequence and glycosylation sufficiently duplicative of that of naturally occurring erythropoietin to allow possession of in vivo biological properties of causing bone marrow cells to increase production of reticulocytes and red blood cells.

The term “erythropoietin isoform” or “EPO isoform” refers to erythropoietin preparations having a single pI, and having the same amino acid sequence. U.S. Pat. No. 5,856,298 describes isoforms of recombinant EPO that differ in extent of glycosylation, e.g., having from 1-14 sialic acids. The EPO isoform can have a desired number (i.e. a fixed number greater than 0) of sialic acids per EPO molecule, with the number of sialic acid residues per EPO molecule ranging from 1-14, with 9, 10, 11, 12, 13, or 14 being of interest, and greater than 14 or 16-23, being of particular interest (see, e.g., U.S. Pat. No. 5,856,298).

“Recombinant EPO” generally refers to an EPO that is the product of the expression of an exogenous DNA sequence that has been transfected into a host cell, particularly a eukaryotic host cell, more particularly a non-human eukaryotic host cell. Recombinant EPO can be produced according to the procedures described in, for example, U.S. Pat. No. 4,703,008. Recombinant EPO can be isolated and purified according to general procedures, such as those described in, for example, U.S. Pat. Nos. 4,667,016, and 5,856,298.

“ARANESP™”, “NESP”, and “darbepoetin alfa” are used interchangeably to refer to an EPO isoform. ARANESP™, which is produced in Chinese hamster ovary (CHO) cells by recombinant DNA technology, is a 165-amino acid protein that differs from recombinant human EPO in containing 5 N-linked oligosaccharide chains, whereas recombinant human erythropoietin contains 3 chains (Egrie et al. Brit J Cancer 2001 84(suppl 1):3-10). The 2 additional N-glycosylation sites result from amino acid substitutions in the EPO peptide backbone. The additional carbohydrate chains increase the approximate molecular weight of the glycoprotein from 30,000 to 37,000 daltons.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and can include: (a) preventing the disease or a symptom of a disease from occurring in a subject which may be predisposed to the disease but has not yet been diagnosed as having it (e.g., including diseases that may be associated with or caused by a primary disease); (b) inhibiting the disease or condition, i.e., arresting its development (e.g., as in inhibiting tumor growth, mitigating or inhibiting worsening of radiation effects upon bone marrow, and the like); and (c) relieving the disease, i.e., causing regression of the disease (e.g., as in facilitating reduction in tumor size, facilitating recovery of bone marrow from radiation, and the like).

“Correction of anemia” refers to providing for recovery of red blood cells in a subject, e.g., as measured by hemoglobin levels in a subject, to a normal range for that subject (e.g., in humans, normal serum hemoglobin levels are about 13-16 g/dL (for women) or about 14-18 g/dL (for men)). “Partial correction of anemia” refers to an increase in red blood cells (e.g., a measured by hemoglobin levels) in a subject compared to prior to initiation of therapy, but where the red blood cell levels in such a subject are still significantly less than that considered within the normal range for that subject (e.g., hemoglobin levels that are less than about 90%, 85%, 80%, 75%, 70%, 65%, 60%, 55%, or 50% of normal or lower).

The terms “individual,” “host,” “subject,” and “patient” are used interchangeably herein, and refer to a mammal, including, but not limited to, primates, including simians and humans.

The term “pharmacokinetic profile,” as used herein, refers to the profile of the curve that results from plotting serum concentration of a drug over time, following administration of the drug to a subject.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a polypeptide” includes a plurality of such polypeptides and reference to “the tumor” includes reference to one or more tumors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention is based on the discoveries that administration of ARANESP™, a recombinant hyperglycosylated variant of EPO, provides for 1) enhanced radiation sensitivity of a tumor in an anemic subject, and 2) mitigation of the effects of, and accelerated recovery from, bone marrow suppression secondary to whole body irradiation of a subject.

In the context of radiotherapy (as in cancer radiotherapy), the present invention is based on the discovery that ARANESP™ enhanced sensitivity of tumors to radiation. The ability of ARANESP™ to potentiate the effects of radiotherapy were not dependent upon either partial correction or complete correction of anemia prior to radiotherapy. Thus, the invention for the first time demonstrates that erythropoietin renders tumors more radiosensitive in a subject who is anemic at the time of radiotherapy. This finding has implications for clinical regimens using ARANESP™ and other erythropoietin polypeptides (such as recombinant erythropoietin (e.g., EPOGEN®)) in radiation therapy for the treatment of tumors in anemic subjects. In short, the present invention indicates that the administration of ARANESP™ is not necessary to correct anemia in a subject prior to radiation therapy.

In addition, administration of ARANESP™ in conjunction with radiation therapy improves the overall health of the subject, e.g., by correction or delaying the undesirable side effects of whole body irradiation, accelerating weight gain after radiotherapy, facilitating recovery of bone marrow suppression associated with radiotherapy, and facilitating recovery of non-erythroid cells (e.g., white blood cells, including neutrophils, lymphocytes, platelets, and the like).

In the context of whole body irradiation, the present invention is based on the discovery that administration of ARANESP™ facilitated bone marrow recovery after exposure of a subject to radiation, and further mitigated the effects of radiation upon bone marrow-derived cells (including non-erythroid cells) and thus serving to protect and/or accelerate recovery of the bone marrow from the effects of radiation. This finding has implications for the use of EPO in treatment or prevention of the effects of intentional or accidental exposure of a subject to radiation.

The invention will now be described in more detail.

EPO and EPO Formulations

Polypeptides having erythropoietin (EPO) activity, particularly those having a desired pharmacokinetic profile are of interest for use in the methods of the present invention. Methods for production of EPO, including EPO isoforms such as ARANESP™, have been described in the art. Furthermore, both EPO and ARANESP™ and formulations of such are commercially available. Methods for production of EPO are described in, for example, U.S. Pat. No. 5,756,349; and 5,955,422. Methods for production of ARANESP™ and other EPO isoforms are described in, for example, U.S. Pat. No. 5,856,298.

EPO polypeptides can be formulated in a variety of ways suitable for administration according to the methods of the invention. In general, EPO polypeptides are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an EPO polypeptides is formulated in an aqueous buffer. Suitable aqueous buffers include, but are not limited to, acetate, succinate, citrate, and phosphate buffers varying in strength from 5 mM to 100 mM. In some embodiments, the aqueous buffer includes reagents that provide for an isotonic solution. Such reagents include, but are not limited to, sodium chloride, and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In the subject methods, the active agents may be administered to the host using any convenient means capable of resulting in the desired therapeutic effect. Thus, the agents can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In general, EPO polypeptides for use in the invention are formulated for parenteral administration, e.g., by subcutaneous, intradermal, intraperitoneal, intravenous, or intramuscular injection. Administration may also be accomplished by, for example, enteral, oral, buccal, rectal, transdermal, intratracheal, inhalation (see, e.g., U.S. Pat. No. 5,354,934), etc.

In pharmaceutical dosage forms, the EPO polypeptide (referred to herein as “the agent” for convenience) may be administered in the form of their pharmaceutically acceptable salts, or they may also be used alone or in appropriate association, as well as in combination, with other pharmaceutically active compounds. The following methods and excipients are merely exemplary and are in no way limiting.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature. Agents can also be provided in sustained release or controlled release formulations, e.g., to provide for release of agent over time and in a desired amount (e.g., in an amount effective to provide for a desired therapeutic or otherwise beneficial effect).

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

ARANESP™ is generally formulated as a sterile, colorless, preservative-free protein solution for intravenous (IV) or subcutaneous (SC) administration. Commercially available single-dose vials contain 25, 40, 60, 100, 150, 200, 300, or 500 micrograms of ARANESP™. Two commercially available formulations contain excipients as follows: 1) Polysorbate solution contains 0.05 mg polysorbate 80, 2.12 mg sodium phosphate monobasic monohydrate, 0.66 mg sodium phosphate dibasic anhydrous, and 8.18 mg sodium chloride in Water for Injection, USP (per 1 mL) at pH 6.2+0.2; and 2) albumin solution contains 2.5 mg albumin (human), 2.23 mg sodium phosphate monobasic monohydrate, 0.53 mg sodium phosphate dibasic anhydrous, and 8.18 mg sodium chloride in Water for Injection, USP (per 1 mL) at pH 6.0±0.3.

It is well within the skill of the ordinary artisan, given the guidance provided herein, to select a dose and dosage regimen of EPO to provide for a desired therapeutic or otherwise beneficial effect in the subject. Precise doses and dosage regimens can vary with such factors as, for example, subject-dependent factors (e.g., body metrics (e.g., weight, height, size, body surface area, and the like), health, tolerance to agent and/or formulation, and the like); agent-dependent factors (e.g., pharmacokinetics (e.g., including serum half-life), bioavailability, and the like); dosage regimen-dependent factors (e.g., route of administration, course of therapy, and the like); and dosage form-dependent factors (e.g., formulation, bolus dosage form, sustained release dosage form, and the like). For example, the serum half-life of ARANESP™ is approximately 3 times longer than that of Epoetin alfa (i.e., PROCRIT® and EPOGEN®). Thus ARANESP™ may be dosed once a week, and in some cases every other week, instead of up to 3 times a week for Epoetin alfa.

For example, effective dosages of ARANESP™ to provide for enhanced radiosensitivity of a tumor in a subject, or to treat, prevent, or mitigate the effects of radiation upon bone marrow in a subject, range from about 0.5 mcg/kg body weight to 4.5 mcg/kg body weight per dose, where the ARANESP™ is to be dosed once weekly, and from about 1.0 mcg/kg body weight to 10 mcg/kg body weight per dose where the drug is to be administered once every two weeks. Exemplary dosages of other EPO polypeptides, e.g., EPOGEN®, can be readily determined by considering the relationship of EPO to ARANESP™. For example, 200 units of Epoetin alfa (EPOGEN®) is equivalent of about 1.0 microgram of ARANESP™. The starting dose of ARANESP™ in the setting of anemia is generally about 0.45 to 0.75 micrograms/kilogram weekly, with subsequence maintenance doses of 0.3 to 0.45 micrograms/kilogram ARANESP™ weekly. Thus EPOGEN® doses may be about 90/kg units to 150 units/kg EPOGEN®, with about 100 units/kg being the average dose, and maintenance doses of EPOGEN®) ranging from about 60 units/kg to 90 units/kg. Due to the shorter half-life of the administered EPO (e.g., EPOGEN®) relative to ARANESP™, dosage regimens may require administration at more frequent intervals (e.g., twice weekly or three times weekly). For example, where ARANESP™ is administered every other week, administration of Epoetin alfa may need to be administered weekly.

Methods of Modulating Effects of Radiation in a Subject

As noted above, the invention is based on the discovery that administration of EPO, as exemplified by the EPO isoform ARANESP™, a recombinant hyperglycosylated variant of EPO, provides for 1) enhanced radiation sensitivity of a tumor in an anemic subject, and 2) mitigation of the effects of, and accelerated recovery from, whole body irradiation of a subject. In addition, administration of EPO provides for improved general health of a subject. Each of these is discussed in more detail below.

Treating a Tumor in an Anemic Host

The invention provides a method of treating a tumor in an anemic host by enhancing the sensitivity of the tumor to radiation through administration of EPO. The methods generally involve administering an effective amount of an EPO polypeptide, in conjunction with radiation therapy of the tumor. “In conjunction” is meant to encompass administration of EPO prior to, at the time of, or subsequent to radiation therapy. In general, the invention does not require that the host receive EPO so that the subject is restored to a non-anemic state, or so as to increase red blood cell count prior to initiation of radiation therapy, e.g., to provide for partial or complete correction of anemia. That is, the methods of the invention do not require administration of EPO in an amount effective to treat anemia in the patient prior to the start of radiation therapy. In general, radiation sensitivity of the tumor is enhanced relative to an expected radiation sensitivity in the absence of EPO administration, and this effect is initially independent of the hemoglobin level.

Tumors susceptible to therapy according to the invention are generally any tumor that, once sensitized according to the invention, can be treated using radiation therapy. Generally, the tumors are solid tumors or lymphohematopoietic tumors. Tumors can be of any tissue origin, grade or stage. For example, the tumors can be associated with cancer of the breast, colon, endometrium, head and neck, lung, skin (e.g., melanoma, basal cell carcinoma, squamous cell carcinoma, and the like), digestive system, gastrointestinal system (including colon, rectum, etc), oral cavity (including lip, mouth, etc.), musculoskeletal system (including muscle, bone, etc.), endocrine system, eye, genitourinary system (e.g., bladder, prostate, ovary, etc.), neurologic system (e.g., brain, etc.), and the like.

The methods of the invention can be used to treat primary tumors or metastases. In addition, the tumors may be either malignant or benign, with radiation therapy generally being used to treat malignant tumors. The invention finds particular use in the treatment of tumors that are generally recognized as radiation resistant (e.g. most solid tumors), and are not highly curable or controllable by radiation therapy. The goal of invention is to sensitize tumors to radiation in order to increase the cure and local control rates of radiation therapy for the treatment of a variety of tumor types.

In some embodiments, an “effective amount” of EPO is an amount that can significantly increase the sensitivity of a tumor to radiation compared to similarly irradiated tumors without EPO treatment (e.g., compared to an expected radiation sensitivity of the tumor type in the absence of EPO administration) (P value <0.05).

Anemic patients generally have serum hemoglobin levels of less than about 13-16 g/dL (for women) or less than about 14-18 g/dL (for men). In general, the invention provides for radiosensitization of one or more tumors in the subject without the need for correction of anemia in the subject prior to initiation of radiation therapy. Anemia is normally said to be “corrected” when hemoglobin levels have returned within the normal range for the sex of the subject or back to the subject's pre-anemia baseline (e.g. following an acute bleed).

EPO, as exemplified by ARANESP™, can be administered as early as 7 days, 11 days, or 18 days prior to initiation of local tumor radiation therapy (either fractionated or single dose therapy, preferably fractionated radiation therapy) or, usually no more than about 7 days prior, generally no more than about 4 days, about 3 days, or about 2 days prior to radiation therapy. Normally EPO is administered within about 1 day prior to or after initiation of radiation therapy, usually prior to initiation of radiation therapy, and can be administered on the same day when radiotherapy is initiated.

Precise dosage regimens will vary according to a variety of factors as discussed above, including in this context the course of radiation therapy prescribed, and, as noted above, the EPO to be administered. Where the EPO is ARANESP™, the EPO is generally administered once every week or every other week during the course of radiation therapy (particularly where radiation therapy is fractionated radiation therapy), although such can be varied according to the needs of the patient. EPO can also be administered after completion of radiation therapy, e.g., for about 1 week, 2 weeks, 3 weeks, or more, particularly if the subject presents with persistent clinically significant anemia.

Radiation therapy can be accomplished according to conventional methods. Radiation can be administered in a single dose or more commonly in fractionated doses, with the latter being of particular interest. For example, fractionated radiation therapy is generally administered daily, 5 days per week for approximately 2 weeks, 4 weeks, 5, weeks, 7 weeks, usually for approximately about 2 to 7.5 weeks, with the specific dosing regimen dependent upon a number of factors including the histological type of tumor and stage of disease.

In one embodiment of the invention, administration of EPO so as to enhance tumor radiosensitivity increases the efficacy of conventional courses of radiation therapy. The invention can, in some embodiments, allow for shortened courses of radiation therapy or reduction in prescribed radiation doses required to achieve a given outcome. For example, administration of EPO in conjunction with radiation therapy may allow the clinician to administer reduced doses of radiation (e.g., about 75%, 80%, or 90-95% of a conventional dose in the absence of EPO therapy), with doses that can be increased or decreased as needed (e.g., relative to the apparent sensitivity of the tumor). Alternatively or in addition, administration of EPO in conjunction with radiation therapy can allow for shortening the course of radiation therapy (e.g., by reducing the total number of treatments required to eradicate a tumor by 1, 2, 3, 4, 5, or more)).

In some embodiments, therapy involves administration of an amount of EPO and radiation dose effective to eradicate the tumor when treating with curative intent or achieve local control/palliate symptoms when treating with palliative intent. For example, therapy according to the invention can involve killing all of the tumor cells in the radiation field or controlling tumor growth (e.g., so as to maintain the tumor at its size at the initiation of therapy), by decreasing or delaying tumor growth.

The effectiveness of EPO in potentiating radiation therapy can be assessed by conventional methods. For example, tumor size (e.g., tumor volume) can be assessed by physical examination in some cases and by the use of imaging techniques such as MRI, CT, ultrasound, PET and the like.

EPO and radiation therapy can be combined with other anti-cancer drugs or treatment regimens as desired, and may be particularly useful in subjects receiving combined modality therapy (either sequential or concurrent chemo and radiation therapy).

Use of EPO in Improving General Health of Subjects Undergoing Radiation Therapy

In addition, the present invention features methods for improving the overall health of a subject undergoing radiation therapy by administration of EPO, particularly ARANESP™. Thus the invention also contemplates administration of EPO in an amount effective to correct or delay undesirable side effects of whole body irradiation, accelerate weight gain of the subject after radiotherapy, facilitate recovery of bone marrow suppression associated with radiotherapy, and/or facilitate recovery of non-erythroid cells (e.g., white blood cells, including neutrophils, lymphocytes, platelets, and the like). EPO therapy in conjunction with radiation therapy can also benefit patients by reducing fatigue and other undesirable side effects of radiation therapy.

Administration of EPO in the context of whole body radiotherapy may also accelerate the return of the subject to at least 90% or at least 95% of body weight prior to initiation of local tumor radiotherapy (e.g. within about 4 days, about 7 days, about 10 days or longer after radiation therapy was initiated).

The invention also features methods for recovery of white blood cells, including neutrophils in anemic subjects, particularly those undergoing radiation therapy. For example, EPO administration can provide for recovery of white blood cells, particularly neutrophils, in anemic subjects to at least about 85%, 90% or 95% of normal levels, usually within about 42 days, about 36 days, about 28 days, about 21 days, about 14 days, about 7 days or less, often usually about 7 days or less, of EPO therapy.

Administration of EPO to Facilitate Recovery of Bone Marrow from Radiation Exposure and/or Mitigate Effects of Radiation Upon Bone Marrow

In one embodiment, the present invention features methods for facilitating bone marrow recovery after exposure of a subject to radiation, as well as to mitigating the effects of radiation upon bone marrow-derived cells (including non-erythroid cells). This latter effect can thus serve to protect the bone marrow from the effects of radiation, e.g., to prevent or mitigate bone marrow suppression associated with radiation exposure.

In this embodiment, EPO, preferably an EPO isoform such as ARANESP™, is administered in an amount effective to treat or prevent/mitigate the effects of intentional or accidental exposure of a subject to radiation, particularly in the context of whole body radiation. Administration of EPO can minimize side effects of radiation exposure; facilitate recovery of bone marrow (e.g., as measured by white blood cell (WBC) recovery, e.g., neutrophil recovery, lymphocyte recovery, platelet recovery, etc.), and/or mitigate the effects of radiation exposure (e.g., by preventing levels of bone marrow-derived cells in the subject from dropping to abnormally low or expectedly low levels after radiation exposure).

EPO can be administered to any subject suffering from, or at risk of radiation exposure, particularly where that radiation exposure may result in bone marrow suppression. “Bone marrow suppression” as used herein refers to reduction in levels of circulating bone marrow derived cells, including non-erythroid cells such as white blood cells, including neutrophils as well as leukocytes (including lymphocytes) and platelets, to a level that is below normal (e.g., normal total leukocytes are about 4,300-10,800/mm³, and neutrophils are about 1,830-7,250/mm³).

EPO can be administered to prevent, mitigate, or treat the effects of radiation exposure that is either accidental or intentional. Such exposures can include those associated with accident (e.g., at nuclear facilities, nuclear waste sites, radiology technicians, and the like), warfare (e.g., conventional or terrorist), or therapy (e.g., as in total body irradiation, e.g., as in bone marrow transplant, and the like). In this embodiment of the invention, the subject need not have a tumor nor be a subject about to undergo cancer therapy (either chemotherapy or radiation therapy). Further, the radiation exposure can be either localized or whole body with the proviso that the radiation exposure is one normally expected to adversely affect bone marrow-derived cells, particular WBC levels, more particularly neutrophil levels, in the subject. In one aspect of particular interest, the radiation is whole body radiation (as compared to localized radiation such as that used in cancer therapy of solid tumors outside the setting of total body irradiation as part of a preparatory regimen for transplantation).

Administration of EPO at the time of or after radiation exposure can facilitate more rapid recovery of WBCs, particularly neutrophils. For example, EPO administration can provide for an increase in (recovery of) neutrophils levels that are at least about 50%, 55%, 60%, 65%, 70%, 75%, 80%, 85%, 90% or 95% of normal levels, usually within about 42 days, about 36 days, about 28 days, about 21 days, about 14 days, about 7 days or less, usually often about 7 days or less, of EPO administration. In general, EPO can be administered within minutes to hours after radiation exposure, or within about 1 day, about 2 days, about 3 days, about 4 days, about 5 days, about 6 days, about 7 days or about 10 days after radiation exposure. In general, EPO should be administered as soon after radiation exposure as possible for best results.

Where EPO is to be administered so as to prevent or mitigate the effects of radiation exposure in a subject about to be exposed to—or at risk of exposure to—radiation, EPO can be administered at about 10 days, about 7 days, about 6 days, about 5 days, about 4 days, about 3 days, about 2 days, or 1 day prior to exposure, or immediately prior to exposure (e.g., within hours to minutes before exposure), with administration within hours or minutes prior to exposure providing for at least some beneficial effects. EPO can then be continued after exposure as above.

Whether administered prior to, at the time of, or after (either immediately or within days) of radiation exposure, the methods of the invention in this embodiment provide for avoiding or mitigating (e.g., providing for partial or complete recovery or prevention of) radiation-induced effects, particularly effects of whole body irradiation. Radiation-induced effects include those associated with acute exposure or delayed effects associated with radiation exposure. Radiation-induced effects may be due to external or internal exposure to radiation.

Acute effects of radiation exposure include, but are not necessarily limited to leucopenia, purpura, hemorrhage, hair loss, diarrhea, fever, electrolyte disturbance, convulsions, ataxia, tremors, and lethargy. EPO administration is useful for treating or preventing those radiation-induced effects related to myelosuppression.

EPO is generally administered to treat subjects exposed to subclinical or sublethal doses of radiation, as defined below. The table below provides a summary of the phases and symptoms of whole body radiation (1 Gray (Gy)=100 rads; 1 centiGray=1 rad).

TABLE
Symptoms of Whole Body Radiation

| | Whole body radiation from external | | | | |
|---|---|---|---|---|---|
| | radiation or internal absorption | | | | |
| | | Subclinical range | | Sublethal range | |
| | | | about 100 | about 200 | about 600 to |
| Phase of | | 0- about | to about | to about | about 800 |
| syndrome | Feature | 100 rad | 200 rad | 600 rad | rad |
| Prodromal | Time of | | 3-6 hrs | 2-4 hrs | 1-2 hrs |
| phase | onset | | | | |
| | Duration | | <24 hrs | <24 hrs | <48 hrs |
| | Lymphocyte | Normal | Minimally | <1000 at | <500 at |
| | count | | decreased | 24 hr | 24 hr |
| Latent | Absence of | >2 wks | 7-15 days | 0-7 days | 0-2 days |
| phase | symptoms | | | | |

| | | | | |
|---|---|---|---|---|
| Acute | Signs and | None | Moderate | Severe leucopenia, |
| Radiation | symptoms | | leucopenia | purpura, hemorrhage |
| Illness or | Time of | | approx. 2 | about 2 days to |
| “manifest | onset | | weeks | about 2 wks |
| illness” | | | | |
| phase | | | | |

*Presented as percent incidence;
**As measured by task performance (TP) and cognitive impairment (CI);
***Critical period - period conventional associated with most potential for effective medical intervention

Examples of delayed radiation-induced effects include, for example, headache, fatigue, weakness, partial and full thickness skin damage, epilation (hair loss), ulceration, anorexia, nausea, vomiting, diarrhea, lymphopenia, neutropenia, thrombopenia, purpura, and opportunistic infections. EPO is useful for treating or preventing those radiation-induced effects related to myelosuppression.

Dosages of EPO in each of these contexts can be based upon the factors as described above. In some embodiments, the EPO dose can be derived from the dose of EPOGEN® recommend for de novo therapy. Doses and dosage regimen for EPO isoforms, such as ARANESP™ can be calculated based on the relationship with EPOGEN® described above. EPO can be administered at intervals as desired, with the number of doses, amount of EPO per dose, and time between doses adjusted as needed to facilitate protection/mitigation and/or recovery.

Kits

Kits with unit doses of a subject EPO-containing formulations suitable for use in enhancing tumor radiosensitivity in an anemic host, e.g., in injectable dose(s), are provided. In such kits, in addition to the containers containing the unit doses will be an informational package insert describing the use and attendant benefits of the drug (e.g., ARANESP™) in treating tumors in conjunction with radiotherapy and, optionally, additional cancer therapeutic agents.

In some embodiments, a subject kit includes a container comprising a solution comprising a unit dose of EPO, particularly ARANESP™, and a pharmaceutically acceptable excipient; and instructions to administer a unit dose according to a desired regiment or exemplary regiment dependent upon tumor type, age, weight, and the like. The instructions can be printed on a label affixed to the container, or can be a package insert that accompanies the container.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Dose-Response Study on Induction of Anemia in C3H Mice by Using Total Body Irradiation (TBI)

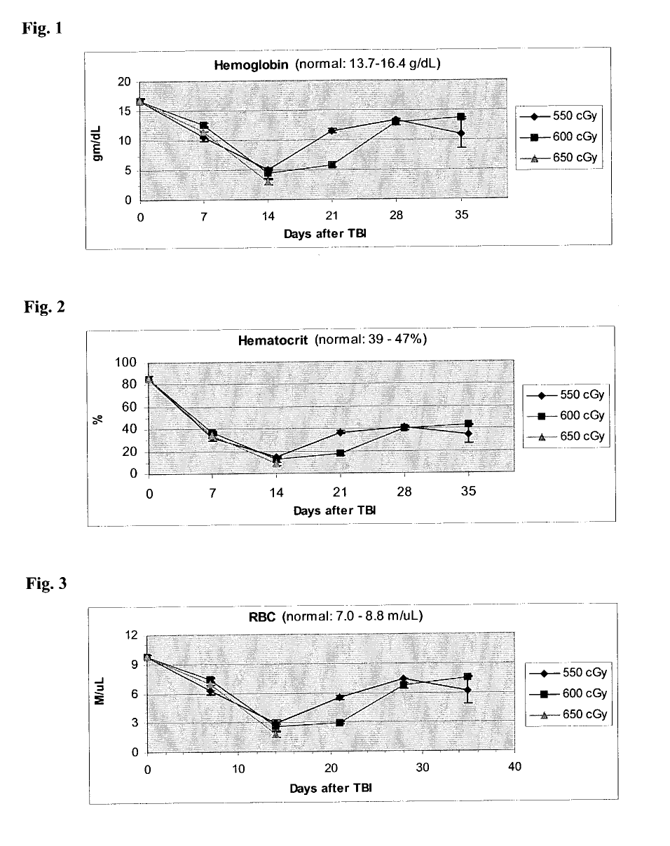

Total-body irradiation (TBI) was used as an anemia-inducing means to establish an anemia model in C3H mice. Normal C3H mice of 8-10 weeks old were irradiated with a single dose of TBI. The levels of hemoglobin (Hgb) and hematocrit (HCT) were used as indicators of anemia. Blood samples were obtained one day before TBI and at 7, 14, 21, 28, and 35 days thereafter. TBI was administered in a single dose at 550 cGy (FIGS. 1-4, diamonds), 600 cGy (FIGS. 1-4, squares) or 650 cGy (FIGS. 1-4, triangles) to C3H male mice (10 weeks old, 25 g in body weight, 4 mice/group).

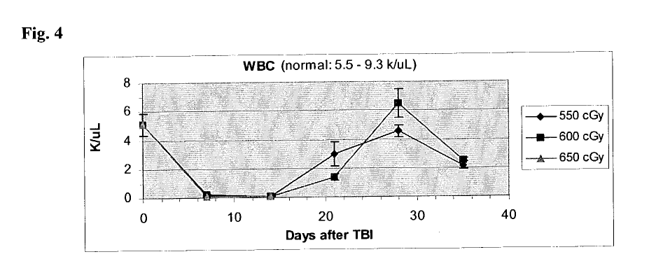

As shown in FIG. 1, a single dose of TBI of 550-650 cGy reduced the hemoglobin level from 15-16 gm/dL (measured one day before TBI) to 3-5 gm/dL on day 14. Hematocrit was reduced from 80-82% at one day before TBI to 9-15% on day 14. The effect of TBI on red blood cell count (RBC) and white blood cell count (WBC) is shown in FIGS. 3 and 4, respectively. One out of 4 mice (25%) in 550 cGy TBI-treated group, 2 mice (50%) in 600 cGy and all 4 mice in 650 cGy group died due to the TBI, suggesting that the TBI dose should be reduced in order to establish a mortality-free stable anemia model.

### Example 2

### Effects of ARANESP™ on TBI-Induced Anemia in C3H Mice

The effects of ARANESP™ on TBI-induced anemia as described in Example 1 was examined. Mice (C3H male mice, 10 weeks old, 25 g in body weight) were divided into the following groups:

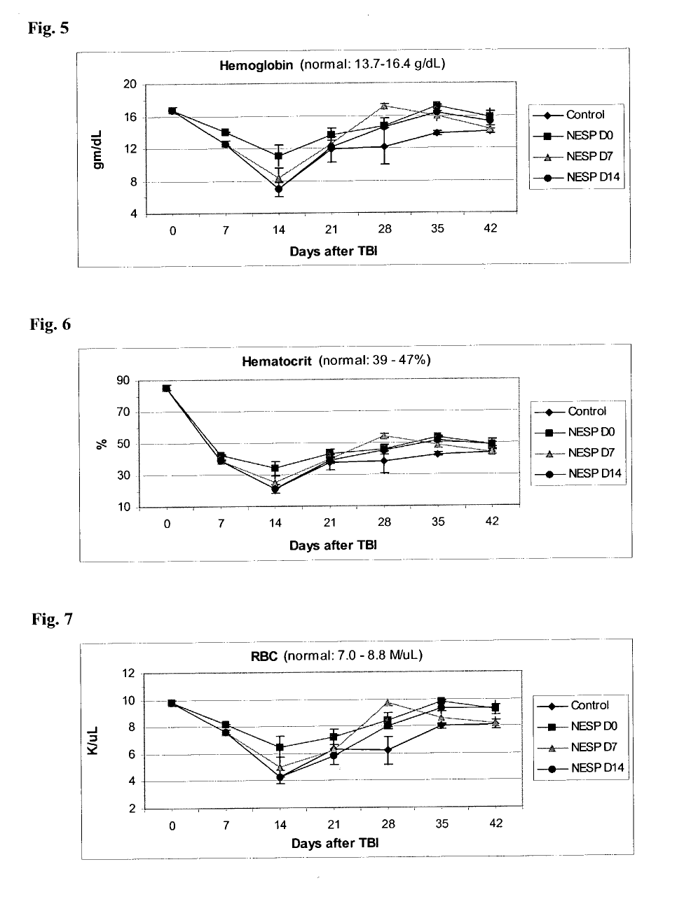

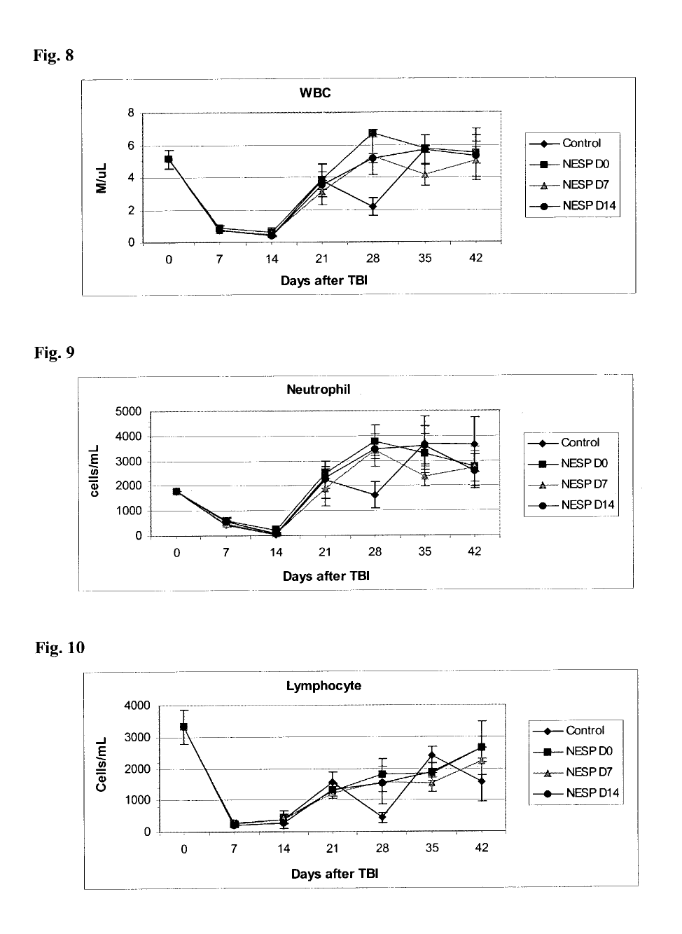

Group 1: Control (TBI only) (FIGS. 5-10, diamonds).

Group 2: ARANESP™ (NESP D0) given immediately following TBI (FIGS. 5-10, squares).

Group 3: ARANESP™ (NESP D7) given 7 days after TBI (FIGS. 5-10, triangles).

Group 4: ARANESP™ (NESP D14) given 14 days after TBI (FIGS. 5-10, circles).

Each group include 8 mice. The TBI dose was 500 cGy. ARANESP™, where applicable, was administered at 30 mcg/kg, i.p., every 2 weeks. Blood samples were collected every two weeks for 4 mice of each group.

The results are provided in FIGS. 5-10. Following a single dose of TBI at 500 cGy, the hemoglobin level in control mice (no NESP) decreased from 15-16 gm/dL to a nadir of 7 gm/dL 14 days after TBI. Hematocrit also decreased in control mice from 64% to 21% at day 14.

ARANESP™ treatment started immediately following TBI (NESP D0) or one week after TBI (NESP D7) significantly reduced the depth and duration of anemia. The hemoglobin was 8.5-11 gram/dL on day 14 in these mice compared to 7 g/dL for mice without ARANESP™ treatment (control). Meanwhile, mice that received ARANESP™ treatment were more healthy and maintained normal levels of activity compared to similarly irradiated mice not treated with ARANESP™.

### Example 3

### Effect of ARANESP™ on Tumor Growth of SCC VII Tumors in TBI-Induced ANEMIC C3H Mice

Fifty C3H mice were randomized into 5 groups with 10 mice per group. All mice were received a single dose of 520 cGy TBI. A murine squamous cell carcinoma, SCC VII, was implanted subcutaneously (s.c.) in all mice 4 hours after TBI. Tumor size measurements were started 12 days after implantation. ARANESP™ was administered by intraperitoneal injection (i.p.) with a dose of 30 mcg/kg at varying times as described below:

Control: Mice were irradiated with TBI (520 cGy), but without ARANESP™ treatment.

N-18: ARANESP™ initiated 18 days before starting tumor measurement (7 days before TBI (−7/TBI)).

N-11: ARANESP™ initiated 11 days before starting tumor measurement (same day as TBI (0/TBI)).

N-4: ARANESP™ was started 4 days before starting tumor measurement (7 days post TBI (7/TBI)).

N0: ARANESP™ was started on the day of starting tumor measurement (II days post TBI (11/TBI)).

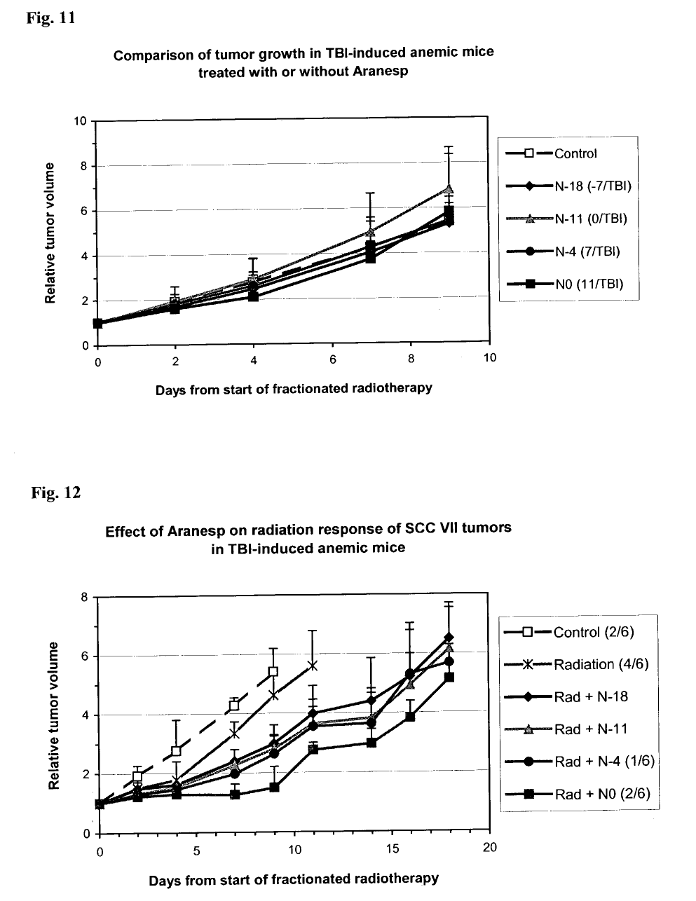

As shown in FIG. 11, administration of ARNESP™ did not alter the tumor growth, that is, ARANESP™ neither promoted or inhibited SCC VII tumor growth in these anemic mice. Administration of ARANESP™ did improve the general health condition of the mice. Mice that were given ARANESP™ before (N-18 group) and on same day (N-11 group) of TBI were the healthiest.

### Example 4

### Effect of ARANESP™ on Radiation Response of SCC VII Tumors in TBI-Induced Anemic Mice

Sixty C3H mice were randomized into 6 groups with 10 mice per group. Mice were irradiated with a single dose of 520 cGy TBI. SCC VII tumors were implanted s.c. 4 hours after TBI. Fractionated radiation therapy (250 cGy/fraction/day for 5 days) was delivered locally 12 days after tumor implantation with an average of tumor volume of 120-150 mm³. ARANESP™ was administered i.p. with a dose of 30 mcg/kg on the designated day and repeated every two weeks. The regimen for each group of mice is provided below (parentheticals in first column refers to symbol used in FIG. 12):

| | |
|---|---|
| Control | Mice were irradiated with TBI (520 cGy), |
| (open | but did not received fractionated |
| squares) | radiotherapy and ARANESP ™. |
| Radiation | Mice were treated with fractionated |
| alone | irradiation (250 cGy/fraction/day × |
| (x's) | 5 days), but without ARANESP ™ treatment. |
| Rad + N- | Mice were treated with fractionated |
| 18 | irradiation and ARANESP ™. ARANESP ™ |
| (diamonds) | was started 18 days before starting |
| | fractionated radiotherapy (7 days |
| | before TBI). |
| Rad + N- | Mice were treated with fractionated |
| 11 | radiation and ARANESP ™. ARANESP ™ |
| (triangles) | treatment was started 11 days before |
| | starting fractionated radiotherapy |
| | (same day with TBI). |
| Rad + N-4 | Mice were treated with fractionated |
| (circles) | radiation and ARANESP ™. ARANESP ™ |
| | treatment was started 4 days before |
| | starting fractionated radiotherapy |
| | (7 days post TBI). |
| Rad + N0 | Mice were treated with fractionated |
| (squares) | radiation and ARANESP ™. ARANESP ™ |
| | treatment was started on same day of |
| | starting fractionated radiotherapy |
| | (11 days post TBI). |

The results are shown in FIG. 12. ARANESP™ enhanced radiation responses of SCC VII tumors. Tumor growth was delayed in mice that received ARANESP™ compared to radiation alone. In addition, administration of ARANESP™ improved the general health condition and reduced the mortality from the bone marrow suppression wrought by TBI and fractionated radiation.

### Example 5

### Effect of ARANESP™ on Radiation Response of SCC VII Tumors in TBI-Induced ANEMIC C3H Mice

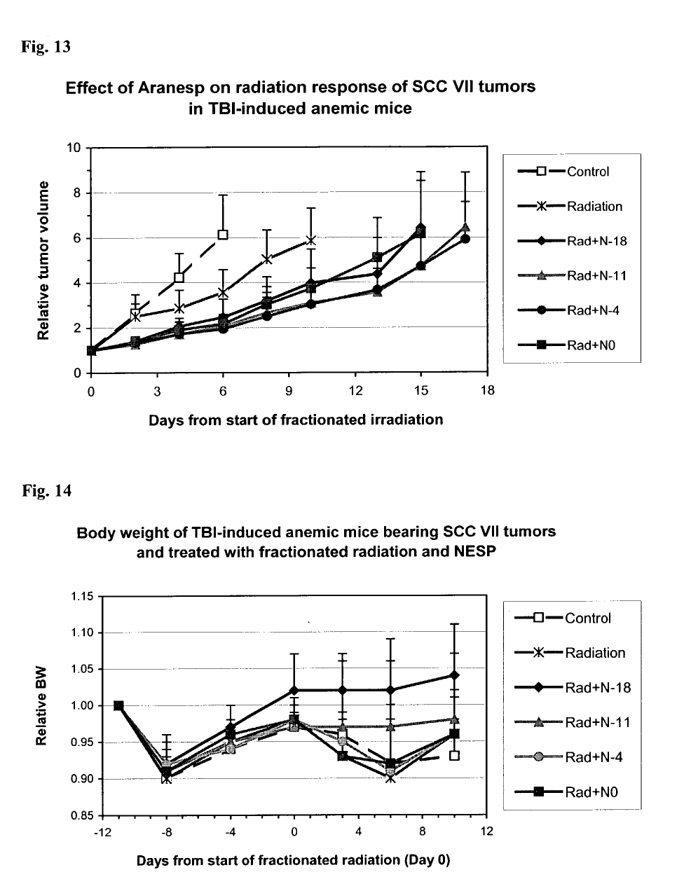

The experiment in Example 4 was repeated using different TBI and ARANESP™ regimen. C3H mice were randomized into 6 groups with 10 mice per group. Mice were irradiated with a single dose of 500 cGy TBI. SCC VII tumors were implanted s.c. 4 hours after TBI. Fractionated radiation therapy (250 cGy/fraction/day for 5 days) was delivered locally 12 days after tumor implantation with an average of tumor volume of 120-150 mm³. Where administered, ARANESP™ was given i.p. with a dose of 30 mcg/kg on the designated day and repeated every two weeks. The regimen for each group of mice is provided below (parentheticals in first column refers to symbol used in FIGS. 13 and 14):

| | |
|---|---|
| Control | Mice were irradiated with TBI (500 cGy), |
| (open squares) | but did not received fractionated |
| | radiotherapy and ARANESP ™. |
| Radiation | Mice were treated with fractionated |
| alone | irradiation (250 cGy/fraction/day × |
| (x's) | 5 days), but without ARANESP ™ |
| | (30 meg/kg, q2w, i.p.).. |
| Rad + N-18 | Mice were treated with fractionated |
| (diamonds) | irradiation and ARANESP ™. ARANESP ™ |
| | was started 18 days before starting |
| | fractionated radiotherapy (7 days |
| | before TBI). |
| Rad + N-11 | Mice were treated with fractionated |
| (triangles) | radiation and ARANESP ™. ARANESP ™ |
| | treatment was started 11 days before |
| | starting fractionated radiotherapy |
| | (same day with TBI). |
| Rad + N-4 | Mice were treated with fractionated |
| (circles) | radiation and ARANESP ™. ARANESP ™ |
| | treatment was started 4 days before |
| | starting fractionated radiotherapy |
| | (7 days post TBI). |
| Rad + N0 | Mice were treated with fractionated |
| (squares) | radiation and ARANESP ™. ARANESP ™ |
| | treatment was started on same day |
| | of starting fractionated radiotherapy |
| | (11 days post TBI). |

The results are shown in FIGS. 13 and 14, and in the table below. ARANESP™ enhanced radiation responses of SCC VII tumors as evidenced by delayed tumor growth compared to radiation alone (P<0.01, FIG. 13 and Table). The greatest effect of ARANESP™ on local tumor responses to the fractionated radiation therapy was observed when ARANESP™ given approximately 7 days before starting the radiation therapy. ARANESP™ also improved the general health condition of TBI-irradiated mice, with accelerated recovery of radiation-induced weight loss (FIG. 14).

TABLE
Comparison of 4X tumor growth.

| | Number | 4X TGT | TGD | P Value (t-test) | | | | |
|---|---|---|---|---|---|---|---|---|
| | of mice | (day) | (day) | Control | RT | R + N0 | R + N − 4 | R + N − 11 |
| Control | 10 | 3.9 ± 1.3 | | | | | | |
| Radiation alone (RT) | 10 | 6.5 ± 1.9 | 2.7 ± 1.9 | 0.002 | | | | |
| RT + ARANESP ™ (0) | 10 | 11.1 ± 3.6 | 7.3 ± 3.6 | 0.00007 | 0.003 | | | |
| RT + ARANESP ™ (−4) | 8 | 13.5 ± 3.0 | 9.7 ± 3.0 | 0.00001 | 0.0001 | 0.1 | | |
| RT + ARANESP ™ (−11) | 6 | 14.5 ± 3.4 | 10.6 ± 3.4 | 0.0004 | 0.001 | 0.1 | 0.6 | |
| RT + ARANESP ™ (−18) | 7 | 11.4 ± 3.5 | 7.5 ± 3.5 | 0.0009 | 0.009 | 0.9 | 0.2 | 0.1 |

*4X TGT (tumor growth time): tumor volume quadrupling time.
**TGD: tumor growth delay time, i.e., 4X TGT of treated tumors minus the mean 4X TGT of control tumors.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of enhancing radiation sensitivity of a tumor in an anemic subject, the method comprising:
administering to the anemic subject an amount of erythropoictin (EPO) effective to enhance radiosensitivity of the tumor relative to the absence of said administering;
wherein radiation sensitivity of the tumor is enhanced prior to or without correction of anemia in the subject.

2. The method of claim 1, wherein EPO is a recombinant EPO having an increased number of sialic residues relative to naturally-occurring EPO.

3. The method of claim 1, wherein EPO is darbepoetin alfa.

4. The method of claim 3, wherein the EPO is administered weekly.

5. The method of claim 4, wherein the EPO is administered in a dose of from about 0.5 mcg/kg body weight to 4.5 mcg/kg body weight.

6. The method of claim 3, wherein the EPO is administered once every two weeks.

7. The method of claim 6, wherein the EPO is administered in a dose of from about 1.0 mcg/kg body weight to 10 mcg/kg body weight.

8. The method of claim 1, wherein EPO is administered prior to said irradiating.

9. The method of claim 1, wherein said irradiating is prior to or at the time of EPO administration.

10. The method of claim 1, wherein the tumor is a solid tumor.

11. A method of treating a tumor in an anemic subject, the method comprising:
administering to an anemic subject having a tumor an amount of erythropoietin (EPO) effective to enhance sensitivity of the tumor to radiation relative to the absence of said administering; and
irradiating the tumor of the anemic subject;
wherein the tumor in the subject is treated.

12. The method of claim 11, wherein EPO is a recombinant EPO having an increased number of sialic residues relative to naturally-occurring EPO.

13. The method of claim 11, wherein EPO is darbepoetin alfa.

14. The method of claim 11, wherein EPO is administered prior to said irradiating.

15. The method of claim 11, wherein said irradiating is prior to or at the time of EPO administration.

16. A method of treating a subject suffering from or at risk of bone marrow suppression associated with radiation exposure, the method comprising:
administering to the subject an amount of erythropoietin (EPO) effective to enhance recovery of red and white blood cell levels in the subject;
wherein said administering is effective to mitigate the effects of radiation upon bone marrow suppression in the subject.

17. The method of claim 16, wherein the radiation exposure is whole body irradiation.

18. The method of claim 16, wherein radiation exposure is accidental.

19. The method of claim 16, wherein EPO is a recombinant EPO having an increased number of sialic residues relative to naturally-occurring EPO.

20. The method of claim 16, wherein the EPO is darpoietin alfa.

21. The method of claim 16, wherein the subject has been exposed to radiation.

22. The method of claim 21, wherein said administering is less than 7 days after radiation exposure.

23. The method of claim 21, wherein said administering is effective to increase white blood cells levels within about 80% of normal levels for the subject.

24. The method of claim 21, wherein said administering is effective to increase neutrophil levels within about 80% of normal level for the subject.

25. The method of claim 16, wherein the subject is at risk of radiation exposure.

26. The method of claim 25, wherein said administering is within about 1 day prior to radiation exposure.

27. The method of claim 25, wherein said administering is effective to mitigate the effects of radiation exposure such that white blood cell levels in the subject are maintained at least about 50% of normal white blood cell levels.

28. The method of claim 25, wherein said administering is effective to mitigate the effects of radiation exposure such that neutrophils levels in the subject are maintained at least about 50% of normal neutrophils levels.
